(19) United States
(12) Patent Application Publication
(10) Pub. No.: US 2016/0367324 A1
(43) Pub. Date: Dec. 22, 2016

(54) INSERTION SHAPE DETECTION APPARATUS

(71) Applicant: OLYMPUS CORPORATION, Tokyo (JP)

(72) Inventors: Ken SATO, Hachioji-shi (JP); Takeshi ITO, Hino-shi (JP); Hiromasa FUJITA, Hachioji-shi (JP)

(73) Assignee: OLYMPUS CORPORATION, Tokyo (JP)

(21) Appl. No.: 15/252,491

(22) Filed: Aug. 31, 2016

Related U.S. Application Data
(63) Continuation of application No. PCT/JP2015/057736, filed on Mar. 16, 2015.

(30) Foreign Application Priority Data
Mar. 20, 2014 (JP) 2014-057657

(51) Int. Cl. A61B 34/20; A61B 1/273; A61B 5/06; A61B 1/307; A61B 1/31; A61B 1/267; A61B 1/303

(52) U.S. Cl. CPC A61B 34/20; A61B 1/267; A61B 1/273; A61B 1/303; A61B 1/307; A61B 1/31; A61B 5/062; A61B 2034/2061; A61B 2034/2048

(57) ABSTRACT

An insertion shape detection apparatus includes an insert portion having flexibility. The insert portion includes a shape estimation section where curved shape is estimated and a shape non-estimation section where curved shape is not estimated. The insertion shape detection apparatus includes a sensing part arranged only in the shape estimation section to detect the curved shape of the shape estimation section. Thus, the number of sensing parts is reduced and the increase in the diameter of the insert portion and complicated processing of curve information is avoided, while the curved shape of the insert portion in a section necessary to assist an endoscopic observation can be detected.

### CROSS-REFERENCE TO RELATED APPLICATIONS

This application is a Continuation Application of PCT Application No. PCT/JP2015/057736, filed Mar. 16, 2015 and based upon and claiming the benefit of priority from prior the Japanese Patent Application No. 2014-057657, filed Mar. 20, 2014, the entire contents of all of which are incorporated herein by references.

### BACKGROUND OF THE INVENTION

1. Field of the Invention

The present invention relates to an insertion shape detection apparatus comprising a flexible insert portion.

2. Description of the Related Art

An insertion shape detection apparatus, for example, an endoscope shape detection apparatus, which comprises a flexible elongated insert portion to be inserted into an insertion target and a sensing part provided in the insert portion to detect a curved shape (a curved angle and a curved direction) of the insert portion, is known,.

For example, Patent Literature 1. Jpn. Pat. Appin. KOKAI Publication No. 2011-200341 discloses an endoscope shape detection apparatus that detects a shape of an insert portion of an endoscope. In the apparatus, a plurality of sensing parts (Fiber Bragg Gratings) are formed on an overall length of an optical fiber extending in a longitudinal direction of the insert portion to detect a shape of the insert portion in its entirety including a soft portion, a curve portion and a distal end portion. The Fiber Bragg Gratings constitute a strain sensor that detects a strain based on a change in wavelength of light at the positions where the gratings are provided in the longitudinal direction of the insert portion, and the curved shape of the insert portion in its entirety is perceived on the basis of the detected strain.

### BRIEF SUMMARY OF THE INVENTION

One embodiment of the invention is an insertion shape detection apparatus comprising an insert portion having flexibility, the insert portion including a shape estimation section where curved shape is estimated and a shape non-estimation section where curved shape is not estimated and a sensing part arranged only in the shape estimation section to detect the curved shape of the shape estimation section. Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing an endoscope system according to a first embodiment of the present invention.

FIG. 2 is a schematic view for explaining a principle of a curved-shape detection sensor.

FIG. 3 is a cross-sectional view taken in a radial direction of an optical fiber for detection light of the curved-shape detection sensor.

FIG. 4 is a schematic view showing organs of a urinary system and an endoscope inserted therein.

FIG. 5 is an enlarged view showing organs of a urinary system and an endoscope inserted therein.

FIG. 6 is a schematic view showing an upper digestive tract and an endoscope inserted therein.

FIG. 7 is a schematic view showing an endoscope system according to variant 1 of the first embodiment of the present invention.

FIG. 8 is a schematic view showing an endoscope system according to variant 2 of the first embodiment of the present invention.

FIG. 9 is a schematic view showing an endoscope system according to variant 3 of the first embodiment of the present invention.

FIG. 10 is a schematic view showing an endoscope system according to a second embodiment of the present invention.

FIG. 11 is a schematic view showing a part of an endoscope system according to the second embodiment of the present invention.

FIG. 12 is a schematic view showing a part of an insertion shape detection apparatus including a catheter.

### DETAILED DESCRIPTION OF THE INVENTION

### [First Embodiment]

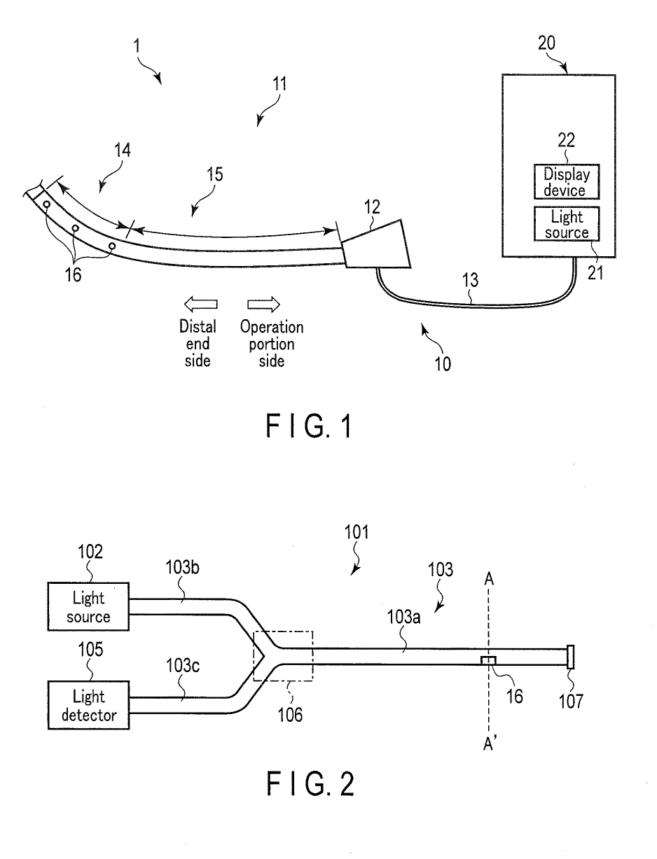

FIG. 1 is a schematic view showing an endoscope system 1 as an insertion shape detection apparatus according to the first embodiment of the present invention. The endoscope system 1 comprises an endoscope 10 and an apparatus main body 20. The endoscope 10 is a living body information obtaining apparatus that observes an inside of an insertion target, for example, a body cavity into which the endoscope is inserted. The apparatus main body 20 comprises a light source 21 that supplies illumination light to the endoscope 10, and a display device 22 that displays an image or the like obtained from the endoscope 10.

The endoscope 10 comprises a flexible insert portion 11 to be inserted in the insertion target, an operation unit 12 coupled to a proximal end side of the insert portion 11, and a cord portion 13 extending from the operation unit 12. The cord portion 13 is detachably connected to the apparatus main body 20, and the endoscope 10 communicates with the apparatus main body 20 via the cord portion 13.

The insert portion 11 is an elongated tubular portion on a distal end side of the endoscope. A distal end portion of the insert portion 11 incorporates an observation optical system including an objective lens; an imaging element that forms an optical image obtained from the observation optical system and converts it into an electric signal; and an illumination optical system including an illumination lens, although not shown in the drawings. An operation wire, a light guide, an electric cable, a channel tube, etc (not shown) are arranged inside the insert portion 11. A curve portion (not shown) on the distal end side of the insert portion 11 is curved in a desired direction by the user' s operation of the operation wire inserted through the insert portion 11 by means of the operation unit 12.

The insert portion 11 includes a shape estimation section 14, which is a section of the distal end side of the insert portion 11 or a section including the distal end, and a shape non-estimation section 15 including a section on a proximal end side of the insert portion 11 (on a side of the operation unit 12) and excluding the shape estimation section 14. A plurality of sensing parts 16 to detect a curved shape of the shape estimation section 14 are arranged in the shape estimation section 14. That is, the sensing parts 16 are arranged only in the shape estimation section 14. Accordingly, the shape estimation section 14 on which the plurality of sensing parts 16 are arranged is a section where curved shape of the insert portion 11 in the section is estimated, whereas the shape non-estimation section 15 on which the sensing parts 16 are not arranged is a section where curved shape of the insert portion 11 in the section is not estimated.

The sensing parts 16 are provided in a curved shape detection sensor 101. FIG. 1 shows only the sensing parts 16 of the curved shape detection sensor 101; however, since an optical fiber 103a for detection light (to be described later) of the curved shape detection sensor 101 is incorporated into the insert portion 11, the curved shape detection sensor 101 is also a component part of the endoscope system 1. The curved shape detection sensor 101 is, for example, a fiber sensor or a strain sensor. In the following, the curved shape detection sensor 101 (hereinafter referred to as the sensor 101) as a fiber sensor is described.

FIG. 2 is a schematic view for explaining a principle of the sensor 101. The sensor 101 comprises a light source 102, an optical fiber 103, and a light detector 105. The optical fiber 103 is connected to the light source 102 and the light detector 105. The light source 102 is, for example, an LED light source or a laser light source which emits detection light having desired wavelength characteristics. The optical fiber 103 transmits the detection light emitted from the light source 102. The light detector 105 detects the detection light guided through the optical fiber 103.

The optical fiber 103 comprises an optical fiber 103a for detection light, an optical fiber 103b for supplying light, and an optical fiber 103c for receiving light, which are branched in three ways at a coupler (optical coupler) 106. Thus, the optical fiber 103 is formed by connecting the optical fiber 103b for supplying light and the optical fiber 103c for receiving light to the optical fiber 103a for detection light by the coupler 106. A proximal end of the optical fiber 103b for supplying light is connected to the light source 102. A reflector (mirror) 107, which reflects the transmitted light, is provided at the distal end of the optical fiber 103a for detection light. A proximal end of the optical fiber 103c for receiving light is connected to the light detector 105.

The optical fiber 103b for supplying light transmits light emitted from the light source 102 and guides it to the coupler 106. The coupler 106 guides large part of light supplied through the optical fiber 103b for supplying light to the optical fiber 103a for detection light, and guides at least part of the light reflected by the reflector 107 to the optical fiber 103c for receiving light. Also, the light from the optical fiber 103c for receiving light is received by the light detector 105. The light detector 105 photoelectrically converts the received detection light, and outputs an electric signal indicative of the amount of detection light.

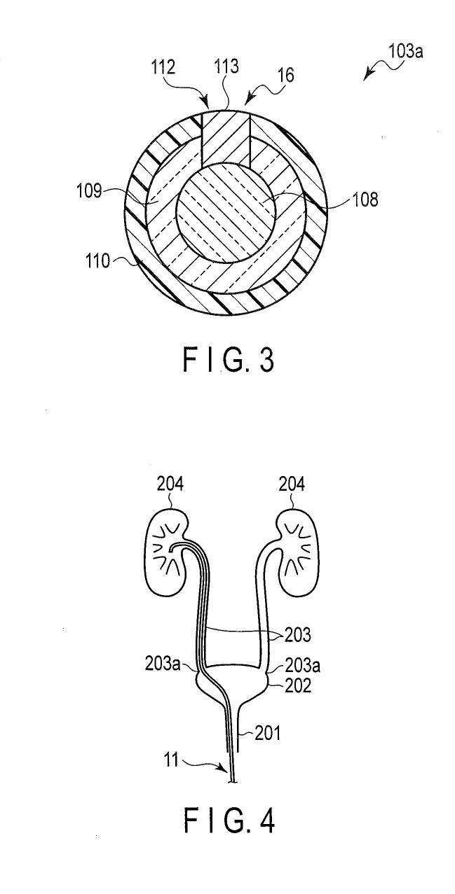

FIG. 3 is a cross-sectional view taken in a radial direction of the optical fiber 103a for detection light, showing a part including a sensing part 16 (the section taken along the line A-A′ in FIG. 2). The optical fiber 103a for detection light comprises a core 108, a cladding 109 that covers an outer periphery of the core 108, and a coating 110 that covers an outer periphery of the cladding 109. The sensing part 16 are formed in the optical fiber 103a for detection light. The sensing parts 16 cause characteristics of light guided through the optical fiber 103a for detection light to change in accordance with a change in curved shape of the sensing parts 16.

The sensing part 16 comprises a light opening 112 which is formed by removing parts of the coating 110 and the cladding 109 to expose the core 108, and a optical characteristic conversion member 113 formed in the light opening 112. The light opening 112 does not necessarily expose the core 108. It is only necessary that the light passing through the optical fiber 103a for detection light reach the optical opening 112. The optical characteristic conversion member 113 is a guide light loss member (light absorber), a wavelength converting member (fluorescent material), or the like, which changes characteristics of the light guided through the optical fiber 103a for light detection. In the following explanation, the optical characteristic conversion member is assumed to be a guide light loss member.

In the sensor 101, the light supplied from the light source 102 is guided through the optical fiber 103a for detection light, as described above. When the light is incident on the optical characteristic conversion member 113 of the sensing part 16, part of the light is absorbed by the optical characteristic conversion member 113, which results in loss of the guided light. The amount of loss of the guided light varies depending on the amount or direction of a curve of the optical fiber 103a for detection light.

For example, even if the optical fiber 103a for detection light is straight, a certain amount of light is lost in the optical characteristic conversion member 113 in accordance with the width of the light opening 112. The amount of loss of light in the straight state is defined as a reference. If the optical characteristic conversion member 113 is located on an outer periphery (outside) of the optical fiber 103a for detection light which is curved, the amount of loss of the guide light is more than the reference amount of lost light. If the optical characteristic conversion member 113 is located on an inner periphery (inside) of the optical fiber 103a for detection light which is curved, the amount of loss of the guide light is less than the reference amount of lost light.

The change of the amount of loss of the guided light is reflected in the amount of detected light received by the light detector 105, that is, the output signal from the light detector 105. Thus, the curved shape (the curved direction and the curved angle) at the position of the sensing part 16 of the sensor 101 is obtained by the output signal from the light detector 105. The optical fiber 103a for detection light is integrally incorporated in the insert portion 11 of the endoscope 10 along the insert portion 11 in the embodiment. The optical fiber 103a for detection light is curved following a curving operation of the insert portion 11, and the sensor 101 detects a curved shape of the insert portion 11 in the shape estimation section 14, as described above. Thus, the curved shape of the insert portion 11 in the shape estimation section 14, including a point (position) whose curved shape is not directly detected at the sensing parts 16 in the shape estimation section 14, is estimated by a computing portion or the like (not shown).

Although FIG. 2 shows only one sensing part 16 in the optical fiber 103a for detection light, a plurality of sensing parts 16 may be provided in different positions in the longitudinal direction of one optical fiber 103a for detection light. Alternatively, the sensor 101 may comprise a plurality of optical fibers 103a for detection light.

The arrangement and length (range) of the shape estimation section 14 of the insert portion 11 are determined on the basis of organs or viscus of an observation target to be observed by the endoscope 10 (an insertion target of the insert portion 11). In the following, a pyeloscope, that is, an endoscope to observe a kidney in the urinary system, will be described as an example.

FIG. 4 is a schematic view showing a urinary system organ and the insert portion 11 of a pyeloscope inserted therein. A tubular urethra 201 leads to a bladder 202 containing a spherical space. The bladder 202 is connected to ureters 203 through right and left ureteral orifices 203a, respectively.

Each ureter 203 is generally a thin tract having an inner diameter of about 3 mm, and leads to a kidney 204 containing a space. In the pyeloscope, the insert portion 11 is inserted through the tubular urethra 201, the bladder 202, an ureteral opening 203a, an ureter 203, and a kidney 204 in this order.

In a tubular organ (tract portion), such as the urethra 201 or the ureter 203, the insert portion 11 is shaped along the shape of the organ. In other words, the insert portion 11 does not significantly change its shape. However, inside the organ containing a space (space portion), such as the bladder 202 or the kidney 204, the insert portion 11 may be shaped in to any shape. Therefore, when the insert portion 11 is inserted into an organ containing a space and the inside of the organ is observed, it is important to determine, for example, to which of the right and left ureteral openings 203a should be directed in the bladder 202, or which calix within the kidney 204 is observed. To make such a determination, it is important to ascertain (detect) the shape of the insert portion 11, in particular, the shape of the distal end of the insert portion 11.

This is why the sensing parts 16 of the sensor 101 are provided in the insert portion 11. However, since the insert portion 11 of the pyeloscope passes through the thin ureter as described above, the diameter of the insert portion 11 needs to be small.

For example, in the case where the curved shape detection sensor is a strain sensor using an electric signal, if a number of sensing parts are provided in the overall length of the insert portion, the number of electric wirings increases accordingly and the resulting configuration will be disadvantageous for reduction in diameter. Alternatively, in the case where the curved shape detection sensor 101 is a fiber sensor, the number of detection points (sensing parts 16) per optical fiber 103a for detection light is limited. Therefore, to provide a number of sensing parts 16 in the overall length of the insert portion 11, a plurality of fiber sensors need be used in a bundle. In this case also, the resulting configuration will be disadvantageous for reduction in diameter.

Therefore, in the embodiment, to avoid increasing the diameter of the insert portion 11, the sensing parts 16 are arranged only in the shape estimation section 14, which is a part of the distal end side of the insert portion 11. Thus, the curved shape of the distal end portion of the insert portion 11 is detected. The number of sensing parts 16 provided in the shape estimation section 14 is limited to ten or less.

The length of the shape estimation section 14 is determined, for example, based on the diameter of the insert portion 11. If the length of the shape estimation section 14 is less than twice the diameter of the insert portion 11, the shape of the insert portion 11 does not significantly change. Therefore, the length of the shape estimation section 14 is equal to or more than twice the diameter of the insert portion 11.

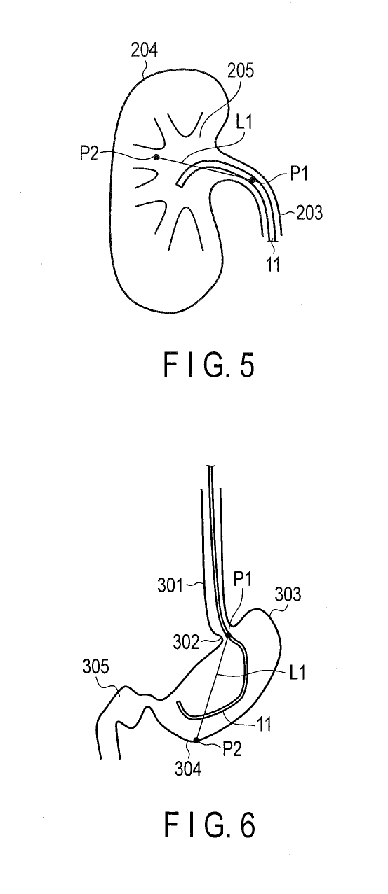

FIG. 5 is an enlarged view showing a urinary system organ and the insert portion 11 of a pyeloscope inserted therein. In the insert portion 11, the length of the shape estimation section 14 is set to be three times or less of a direct distance L1 from a starting point P1 of a space inside the living body (a point from which the ureter 203 starts to extend to a renal pelvis 205 in FIG. 5) to a farthest point P2 of an observation range. Even if the spherical space extends from the starting point P1 of the space inside the living body to the farthest point P2 of the observation range, it is enough to ascertain the shape of the insert portion 11 in the space inside the living body if setting the length of the shape estimation section 14 to about three times (an approximate circular constant) the direct distance L1 from the starting point P1 of the space inside the living body to the farthest point P2 of the observation range. For example, in the case of a pyeloscope, the length of the shape estimation section 14 is preferably set to be within 0.5 cm to 10 cm.

The embodiment is particularly suitable for an endoscope for observing an organ which contains a space (space portion) extending from a thin insert path (tube portion). The organ which contains a space extending from a thin insert path may be a stomach or a duodenum in the digestive system, in addition to the kidney in the urinary system described above.

FIG. 6 is a schematic view showing an upper digestive tract and the insert portion 11 of an upper digestive tract scope inserted therein. For the insert portion 11 of the upper digestive tract scope inserted into an upper digestive tract (stomach 303 or duodenum 305) through an esophagus 301, the length of the shape estimation section 14 is set to be within 2 cm to 60 cm. For example, if the observation target is the stomach 303, the starting point P1 of the space inside the living body is a cardiac 302 and the farthest point P2 of the observation range is a vestibule 304.

As described above, in the embodiment, the shape of the insert portion 11 in the shape estimation section 14 is detected by means of the sensing parts 16 arranged in the shape estimation section 14, which is a section of the distal end side of the insert portion 11 or a section including the distal end. Thus, the curved shape of a distal end portion of the insert portion 11 is ascertained.

According to the embodiment, the sensing parts are provided only in the shape estimation section of the insert portion of the endoscope. As a result, the number of sensing parts is reduced and the increase in the diameter of the insert portion and complicated processing of curve information is avoided, while the curved shape of the insert portion in a section necessary to assist an endoscopic observation can be detected. Thus, it can provide a convenient shape detection apparatus.

(Variant 1)

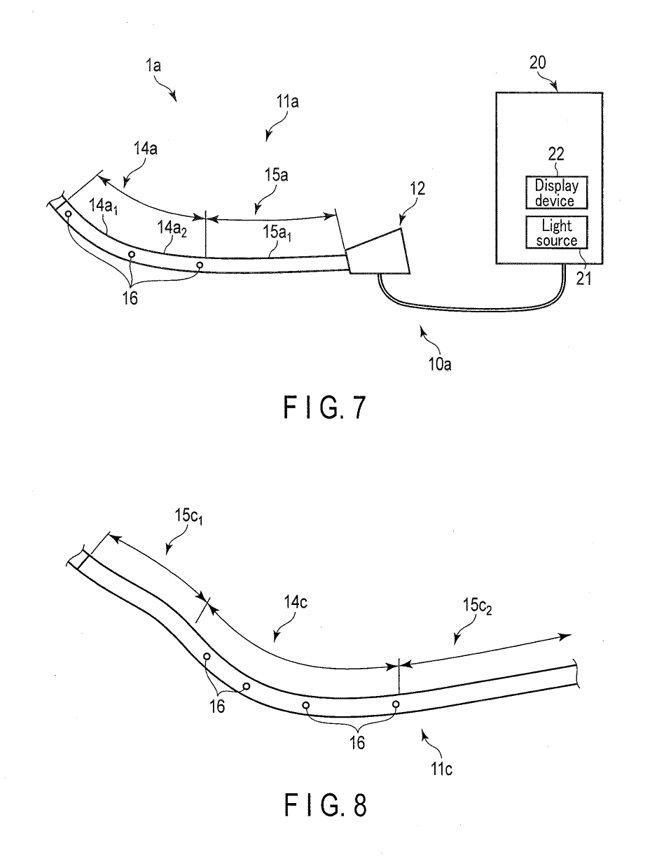

FIG. 7 is a schematic view showing an endoscope system according to variant 1 of the first embodiment of the present invention. The endoscope system 1 comprises an endoscope 10a and an apparatus main body 20. The endoscope 10a comprises a flexible insert portion 11a, an operation unit 12, and a cord portion 13. In the variant, the insert portion 11a comprises an active curve portion 14a₁ and a passive curve portion 14a₂ in a distal end side, and a soft portion 15a₁ in a proximal end side.

The active curve portion 14a₁ is flexible and curved by operating an operation wire (not shown) inserted through the insert portion 11a by means of the operation unit 12. The passive curve portion 14a₂ is coupled to a proximal end side of the active curve portion 14a₁. The passive curve portion 14a₂ is also flexible. However, the passive curve portion 14a₂ is not curved by means of the operation unit 12.

The passive curve portion 14a₂ is more flexible and more bendable than the soft portion 15a₁ that is coupled to its proximal end side. Therefore, when the passive curve portion 14a₂ is brought into contact with an inner wall of a lumen as a target of insertion, it bends sooner than the soft portion 15a₁. Thus, the active curve portion 14a₁ and the passive curve portion 14a₂ form a section which easily changes shape. Therefore, in the variant, the active curve portion 14a₁ and the passive curve portion 14a₂ are set as a shape estimation section 14a.

Although the soft portion 15a₁ is flexible, it is less flexible and less bendable as compared to the passive curve portion 14a₂. Furthermore, the soft portion 15a₁ is a portion which cannot be curved by means of the operation unit 12. In the variant, the soft portion 15a₁ is set as a shape non-estimation section 15a.

Thus, in the variant, the active curve portion 14a₁ and the passive curve portion 14a₂ are set as the shape estimation section 14a, and sensing parts 16 are arranged in the curve portions, respectively. A curved shape of the insert portion 11 in the shape estimation section 14a is detected by means of the sensing parts 16.

According to the variant, a section near the distal end of the insert portion 11a, which easily changes its shape, is set as the shape estimation section. Therefore, a possible change in shape can be ascertained more reliably and appropriately.

(Variant 2)

FIG. 8 is a schematic view showing an endoscope system according to variant 2 of the first embodiment of the present invention. An insert portion 11c comprises a shape estimation section 14c, a first shape non-estimation section 15c₁, and a second shape non-estimation section 15c₂. The shape estimation section 14c is interposed between the first shape non-estimation section 15c₁ and the second shape non-estimation section 15c₂.

In a case of observing an organ or an internal organ having a branched insert path, for example, a respiratory organ, it is important to ascertain in which direction the insert portion is inserted through the branch of the windpipe. Therefore, as in the variant, it is useful to locate the shape estimation section between the two shape non-estimation sections. If the target of the observation is a respiratory organ, the length of the shape estimation section is set to be within 0.5 cm to 30 cm, based on the same point of view for setting the length of the shape estimation section for a kidney and an upper digestive tract as described above.

Furthermore, in a case of observing an organ which can flexibly deforms, for example, a lower digestive tract (such as a large intestine), it is important to ascertain whether the insert portion unnecessarily bends in the large intestine and makes the insertion difficult. In this case also, it is useful to locate the shape estimation section between the two shape non-estimation sections, so that a curved shape in a middle part of the insert portion can be detected. Moreover, if the target of observation is a lower digestive tract, the length of the shape estimation section is set to be within 2 cm to 100 cm in the same manner.

According to the variant, the shape estimation section 14c is interposed between an operation unit and a distal end portion of the insert portion 11c . As a result, the shape of a middle part of the insert portion 11c can be ascertained. In FIG. 8, the number of shape estimation section 14c is one, but may be two or more.

The following shows the relationship between an insertion target and a length of the shape estimation section of the insert portion of the endoscope according to the embodiment and the variant.

TABLE 1

| Insertion target | Length of shape detection section |
|---|---|
| Kidney | 0.5 cm to 10 cm |
| Bladder | 1 cm to 15 cm |
| Upper digestive tract | 2 cm to 60 cm |
| Lower digestive tract | 2 cm to 100 cm |
| Respiratory organ | 0.5 cm to 30 cm |
| Female reproductive organ | 2 cm to 60 cm |

The ratio between the shape estimation section 14 and the shape non-estimation section 15 can be determined on the basis of, for example, the ratio between the tube portion and the space portion of an insertion target. Furthermore, the length of the shape estimation section 14 may be set to be, for example, shorter than the length of the shape non-estimation section 15. Furthermore, the length of the shape estimation section 14 may be set to 50 times or less of the diameter of the insert portion 11. The setting described above can provide an insertion shape detection apparatus which is convenient and suitable for a thin insert portion without complicated processing of curve information.

(Variant 3)

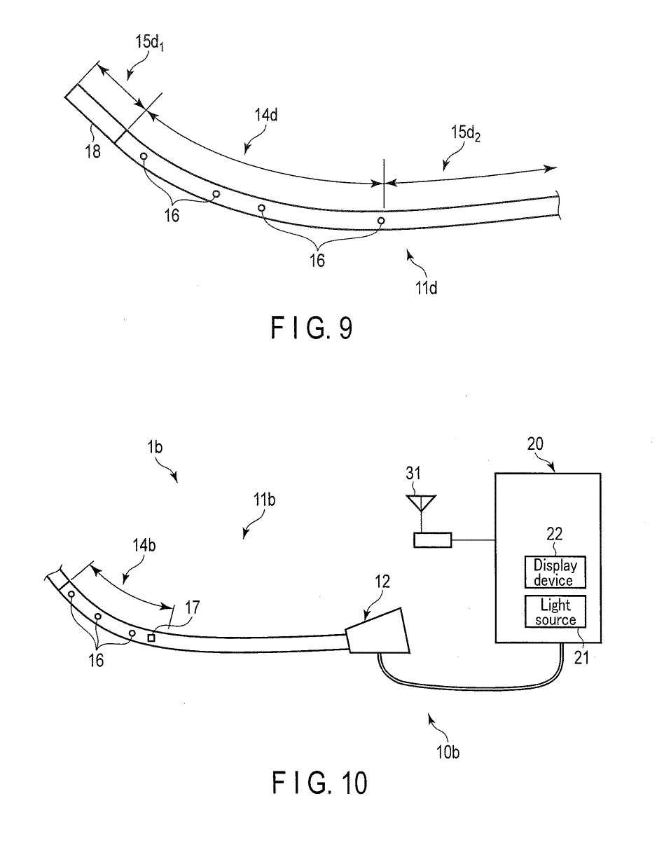

FIG. 9 is a schematic view showing an endoscope system according to variant 3 of the first embodiment of the present invention. In the variant, an insert portion 11d is flexible, except for a part. The excepted part is a distal end hard portion 18, which incorporates an observation optical system, an illumination optical system, an imaging element, etc. near the distal end of the insert portion. The distal end hard portion 18 is hard and unbendable. In other words, the distal end hard portion 18 does not change its shape.

The insert portion 11d comprises a shape estimation section 14d, a first shape non-estimation section 15d₁ and a second shape non-estimation section 15d₂, as well as variant 2. The shape estimation section 14d is interposed between the first shape non-estimation section 15d₁ and the second shape non-estimation section 15d₂. In the variant, the first shape non-estimation section 15d₁ is the distal end hard portion 18.

In the variant, the part that does not change its shape is set to a shape non-estimation section, so that the number of sensing parts 16 can be reduced.

### [Second Embodiment]

The second embodiment of the present invention will be explained with reference to FIG. 10 to FIG. 12 In the following, the same reference numeral as used in the first embodiment will be used for the same, and detailed descriptions thereof will be omitted and only matters different from the first embodiment will be explained.

The second embodiment is an endoscope system 1b as an insertion shape detection apparatus in which the sensing parts 16 and detection of at least one of a position and an orientation are combined.

The endoscope system 1b comprises an endoscope 10b including a flexible inert portion 11b, an apparatus main body 20, and a position and orientation detector 31. The position and orientation detector 31 is illustrated as being independent of the apparatus main body 20, but may be incorporated into the apparatus main body 20.

In the embodiment, a position and orientation marker 17 as an additional sensing part is provided in a shape estimation section 14b of the insert portion 11b. The position and orientation marker 17 comprises, for example, an acceleration sensor or a magnetic coil. If a plurality of position and orientation markers 17 is provided, at least one of the position and orientation markers 17 may be located in the shape estimation section 14b. The position and orientation detector 31 detects at least one of the position and the orientation of the position and orientation marker 17.

According to the embodiment, the sensing part 16 serves to ascertain a shape of the shape estimation section 14b of the insert portion 11b. In addition, the position and orientation marker 17 serves to reliably and appropriately ascertain in what position and orientation the shape estimation section 14b is inserted in the space. Moreover, since it is possible to detect where in the space the insert portion is inserted and what shape the distal end of the insert portion has, the convenience of the operation of the endoscope can be improved.

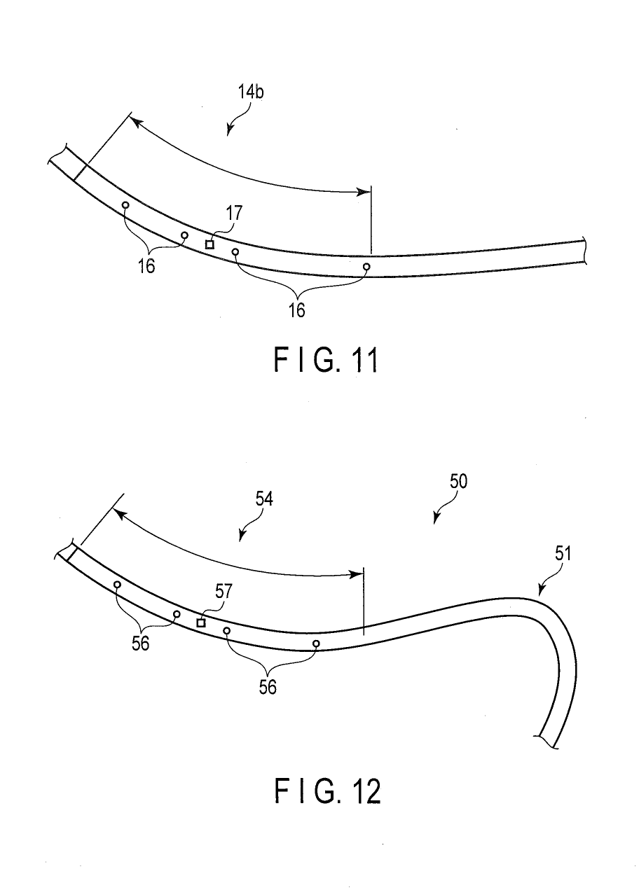

In FIG. 10, the position and orientation marker 17 is located in the shape estimation section 14b in a side of the operation unit; however, it may be located in a distal end side, or a central portion of the shape estimation section as shown in FIG. 11.

Although the endoscope having a flexible insert portion has been described above as an example, the insertion shape detection apparatus of the present invention is applicable to not only endoscopes, but anything that has an insert portion to be inserted into a target in use as long as the insert portion is flexible. For example, targets of application may be medical or industrial endoscopes, catheters, forceps, etc.

FIG. 12 is a schematic view showing a part of an insertion shape detection apparatus including a catheter 50. The catheter 50 comprises a flexible insert portion 51, which is inserted into an insertion target. The insert portion 51 includes a shape estimation section 54, which is a section of the distal end side of the insert portion 51 or a section including the distal end. In FIG. 12, reference numeral which represents the shape non-estimation section is not referred; however, the section other than the shape estimation section 54 in the insert portion 51 is the shape non-estimation section. Sensing parts 56 are arranged in the shape estimation section 54. Furthermore, a position and orientation marker 57 may also be arranged in the shape estimation section 54.

In the insertion shape detection apparatus comprising a catheter as described above, the number of sensing parts is reduced and the increase of the diameter of the insert portion and complicated processing of curve information is avoided, while the curved shape of the insert portion in a section where the curved shape should be ascertained can be appropriately and reliably detected. Thus, it can provide a convenient shape detection apparatus.

The present invention is not limited to the foregoing embodiment described above, but it is evident to a person with ordinary skill in the art that various improvements and modifications can be made without departing from the subject matter of the present invention.

### REFERENCE SIGNS LIST

1 . . . Endoscope system, 10, 10a, 10b . . . Endoscope, 11, 11a, 11b . . . Insert portion, 12 . . . Operation portion, 13 . . . Cord portion, 14, 14a, 14b, 14c, 14d . . . Shape estimation section, 14a₁ . . . Active curve portion, 14a₂ . . . Passive curve portion, 15, 15a, 15c₁, 15c₂, 15d₁, 15d₂ . . . Shape non-estimation section, 15a₁ . . . Soft portion, 16 . . . Sensing part, 17 . . . Position and orientation marker, 18 . . . Distal end hard portion, 20 . . . Apparatus main body, 21 . . . Light source, 22 . . . Display device, 31 . . . Position and orientation detector, 50 . . . Catheter, 51 . . . Insert portion, 54 . . . Shape estimation section, 56 . . . Sensing part, 57 . . . Position and orientation marker, 101 . . . Curved shape detection sensor, 102 . . . Light source, 103 . . . Optical fiber, 103a . . . Optical fiber for detection light, 103b . . . Optical fiber for supplying light, 103c . . . Optical fiber for receiving light, 105 . . . Light detector, 106 . . . Coupler, 107 . . . Reflector, 108 . . . Core, 109 . . . Cladding, 110 . . . Coating, 112 . . . Light opening, 113 . . . Optical characteristic conversion member, 201 . . . Tubular urethra, 202 . . . Bladder, 203 . . . Ureter, 203a . . . Ureteral opening, 204 . . . Kidney, 205 . . . Renal pelvis, 301 . . . Esophagus, 302 . . . Cardia, 303 . . . Stomach, 304 . . . Vestibule, 305 . . . Duodenum.

What is claimed is:

1. An insertion shape detection apparatus comprising:
an insert portion having flexibility, the insert portion including a shape estimation section where curved shape is estimated and a shape non-estimation section where curved shape is not estimated; and
a sensing part arranged only in the shape estimation section to detect the curved shape of the shape estimation section.

2. The insertion shape detection apparatus according to claim 1, wherein a length of the shape estimation section is determined based on an insertion target into which the insert portion is to be inserted.

3. The insertion shape detection apparatus according to claim 1, wherein an insertion target into which the insert portion is to be inserted includes a tube portion and a space portion.

4. The insertion shape detection apparatus according to claim 3, wherein the insertion target is one of a kidney, a bladder, an upper digestive tract and a female reproductive organ.

5. The insertion shape detection apparatus according to claim 3, wherein a ratio between the shape estimation section and the shape non-estimation section is determined based on a ratio between the tube portion and the space portion.

6. The insertion shape detection apparatus according to claim 3, wherein the insertion target is a kidney and the length of the shape estimation section is 0.5 to 10 cm.

7. The insertion shape detection apparatus according to claim 3, wherein the insertion target is a bladder and the length of the shape estimation section is 1 to 15 cm.

8. The insertion shape detection apparatus according to claim 3, wherein the insertion target is an upper digestive tract and the length of the shape estimation section is 2 to 60 cm.

9. The insertion shape detection apparatus according to claim 3, wherein the length of the shape estimation section is three times or less of a distance from a starting point to a farthest point of the space portion.

10. The insertion shape detection apparatus according to claim 1, wherein an insertion target into which the insert portion is to be inserted is a tube portion.

11. The insertion shape detection apparatus according to claim 10, wherein the insertion target into which the insert portion is to be inserted is one of a respiratory organ and a lower digestive tract.

12. The insertion shape detection apparatus according to claim 11, wherein the insertion target is a respiratory organ and the length of the shape estimation section is 0.5 to 30 cm.

13. The insertion shape detection apparatus according to claim 11, wherein the insertion target is a lower digestive tract and the length of the shape estimation section is 2 to 100 cm.

14. The insertion shape detection apparatus according to claim 1, wherein the length of the shape estimation section is equal to or shorter than the length of the shape non-estimation section.

15. The insertion shape detection apparatus according to claim 1, wherein the length of the shape estimation section is no more than 50 times of a diameter of the insert portion.

16. The insertion shape detection apparatus according to claim 1, wherein the insert portion comprises a soft portion, a passive curve portion and an active curve portion, and the passive curve portion and the active curve portion are located in the shape estimation section.

17. The insertion shape detection apparatus according to claim 16, wherein the sensing parts are provided in each of the passive curve portion and the active curve portion.

18. The insertion shape detection apparatus according to claim 1, wherein the sensing parts are provided in a fiber sensor.

19. The insertion shape detection apparatus according to claim 1, wherein the shape estimation section is arranged in a distal end side of the insert portion.

20. The insertion shape detection apparatus according to claim 1, comprising a plurality of shape non-estimation sections, and characterized in that the shape estimation section is arranged between the shape non-estimation sections.

21. The insertion shape detection apparatus according to claim 1, further comprising an additional sensing part to detect at least one of a position and an orientation in the shape estimation section.

22. The insertion shape detection apparatus according to claim 21, wherein the additional sensing part is a position and orientation marker including a magnetic coil, and characterized by further comprising a position and orientation detector to detect a position and an orientation of the position and orientation marker.

23. The insertion shape detection apparatus according to claim 21, wherein the additional sensing part comprises an acceleration sensor.

24. The insertion shape detection apparatus according to claim 1, wherein the number of the sensing parts is ten or less.
